(19) United States
(12) Patent Application Publication
(10) Pub. No.: US 2012/0283550 A1
(43) Pub. Date: Nov. 8, 2012

(54) HEAD SUPPORT CUSHION WITH INTEGRATING PATIENT POSITIONING SYSTEM

(76) Inventor: Daniel Driemel, Oederan (DE)

(21) Appl. No.: 13/465,023

(22) Filed: May 6, 2012

(30) Foreign Application Priority Data
May 6, 2011 (DE) DE 102011075440.7

(51) Int. Cl. A61B 5/055

(52) U.S. Cl. 600/415

(57) ABSTRACT

A local coil for an imaging system includes a support cushion including a volume filled with a fluid.

This application claims the benefit of DE 10 2011 075 440.7, filed on May 6, 2011.

### BACKGROUND

The present embodiments relate to a local coil.

Magnetic resonance devices (MRTs) for examining objects or patients using magnetic resonance tomography are known, for example, from DE10314215B4.

In MR tomography, images with a high signal to noise ratio (SNR) may be recorded using local coils. The local coils are antenna systems that are positioned in direct proximity on (anterior) or beneath (posterior) the patient. During an MR measurement, the excited nuclei induce a voltage in the individual antennas of the local coil. The induced voltage is amplified using a low-noise preamplifier (LNA, preamp) and forwarded by cable to the electronic receive system. High-field systems (e.g., 1.5 T to 12 T and more) are used to improve the signal to noise ratio, even with high-resolution images. Since more individual antennas may be connected to an MR receive system than there are receivers present, a switching matrix (e.g., RCCS) is incorporated between receive antennas and receivers. This routes the currently active receive channels (e.g., the receive channels in the field of view of the magnet at the time) to the receivers present. This allows more coil elements to be connected than there are receivers present, since for whole body coverage, the coils present in the field of view (FoV) or homogeneity volume of the magnet are to be read out.

A coil (or local coil) may, for example, be an antenna system that may include one or more antenna elements (e.g., coil elements; an array coil).

The individual antenna elements may be embodied as loop antennas or loops, butterfly coils, or saddle coils. A coil includes the coil elements, the preamplifier, further electronic elements (e.g., baluns) and cabling, the housing and may include a cable with plug that may be used to connect the coil to the MR system. A receiver (RX) on the MRT system side filters and digitizes signals received by the local coil and transfers the data to the digital signal processor. The digital signal processor may derive an image or spectrum from the measurement, making the image or spectrum available to the user for diagnosis.

To achieve optimum image quality, the patient lies still during an MR measurement, without changing position in or on the respective MR coil. Additional patient positioning measures are provided in addition to the application of the respective MR coil. For example, during head examinations the patient may be positioned in a stable manner to prevent head movements caused, for example, by breathing or swallowing.

According to a known solution, the head of the patient is positioned on a foam part in a head coil lower part during head examinations. This cushion cushions the rear of the head of the patient to prevent pressure points due to the fixed housing of the lower part of the head coil. The cushion is flat, so that the patient has space at the side to position the patient in the coil. Head movement to the left and right is restricted, for example, using additional cushions that are positioned between the coil housing and the head of the patient.

Clamping jaws may also be used with head coils with a smaller number of channels. The clamping jaws pass to the left and right through openings in the coil housing and are pushed against the head on both sides until the head is clamped in place, thereby preventing lateral movement of the head.

### SUMMARY

The present embodiments may obviate one or more of the drawbacks or limitations in the related art. For example, a local coil for an imaging system may be optimized.

### BRIEF DESCRIPTION OF THE DRAWINGS

FIG. 1 shows a perspective view of one embodiment of a cushion for one embodiment of a local coil;

FIG. 2 shows a perspective view of one embodiment of a cushion in a lower part of one embodiment of a local coil;

FIG. 3 shows a perspective view of one embodiment of a cushion in the lower part of one embodiment of a local coil with a head on the cushion;

FIG. 4 shows a perspective view of one embodiment of a cushion in the lower part of one embodiment of a local coil with a head on the cushion and with a local coil upper part of the local coil positioned on the local coil lower part of the local coil; and

FIG. 5 shows a schematic diagram of a magnetic resonance tomography (MRT) system.

### DETAILED DESCRIPTION OF THE DRAWINGS

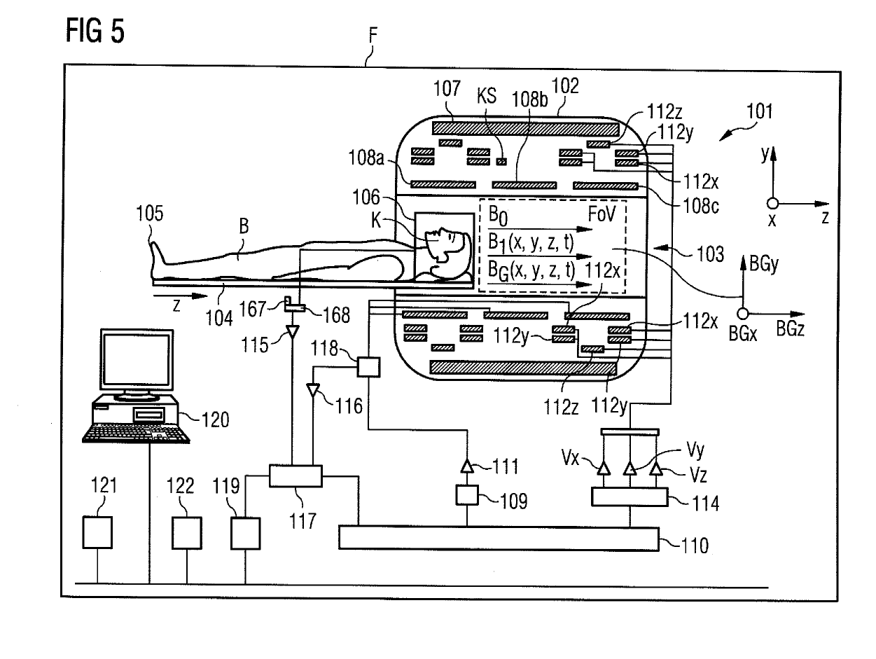

FIG. 5 shows a magnetic resonance imaging device MRT 101 (e.g., present in a screened chamber or Faraday cage F) with a whole body coil 102. The whole body coil 102 includes a, for example, tubular chamber 103, into which a patient couch 104 holding a body 105 (e.g., of an examination object such as a patient) may be moved (e.g., with or without local coil arrangement 106) in the direction of the arrow z, in order to generate recordings of the patient 105 using an imaging procedure. Disposed on the patient, for example, is the local coil arrangement 106, which may be used in a local region (e.g., a field of view (FoV)) of the magnetic resonance imaging device MRT 101 to generate recordings of a subregion of the body 105 in the FoV. Signals from the local coil arrangement 106 may be analyzed by an analysis device or facility (e.g., including elements 168, 115, 117, 119, 120, 121) of the MRT 101 that may be connected, for example, by way of coaxial cable or a radio (e.g., element 167) to the local coil arrangement 106 (e.g., being converted to images, stored or displayed).

In order to use a magnetic resonance device MRT 101 to examine a body 105 (e.g., an examination object or patient) using magnetic resonance imaging, different magnetic fields, temporal and spatial characteristics of which are matched very precisely to one another, are radiated onto the body 105. A powerful magnet (e.g., a cryomagnet 107) in a measuring cabin with a, for example, tunnel-shaped opening 103, generates a powerful static main magnetic field B₀ of, for example, 0.2 Tesla to 3 Tesla or more. The body 105 to be examined is supported on a patient couch 104 and moved into a roughly homogeneous region of the main magnetic field B₀ in the FoV. Excitation of the nuclear spin of atomic nuclei of the body 105 takes place by way of magnetic high-frequency excitation pulses B1(x, y, z, t) that are radiated in by way of a high-frequency antenna shown in FIG. 5 in a simplified manner as a body coil 108 (e.g., a multipart body coil 108a, 108b, 108c; and/or optionally a local coil arrangement). High-frequency excitation pulses are generated, for example, by a pulse generation unit 109 that is controlled by a pulse sequence control unit 110.

After amplification by a high-frequency amplifier 111, the high-frequency excitation pulses are routed to the high-frequency antenna 108. The high-frequency system illustrated in FIG. 5 is only shown schematically. In some embodiments, more than one pulse generation unit 109, more than one high-frequency amplifier 111 and a number of high-frequency antennas 108 a, b, c are used in a magnetic resonance device 101.

The magnetic resonance device 101 also has gradient coils 112x, 112y, 112z, that are used during a measurement to radiate in magnetic gradient fields for selective slice excitation and for local coding of the measurement signal.

The gradient coils 112x, 112y, 112z are controlled by a gradient coil control unit 114 that, like the pulse generation unit 109, is connected to the pulse sequence control unit 110.

Signals emitted by the excited nuclear spin (e.g., of the atomic nuclei in the examination object) are received by the body coil 108 and/or at least one local coil arrangement 106, amplified by assigned high-frequency preamplifiers 116 and further processed and digitized by a receive unit 117. The recorded measurement data is digitized and stored as complex numerical values in a k-space matrix. A multi-dimensional Fourier transformation may be used to reconstruct an associated MR image from the value-populated k-space matrix.

For a coil that may be operated in both transmit and receive mode (e.g., the body coil 108 or a local coil 106), correct signal forwarding is regulated by an upstream duplexer 118.

An image processing unit 119 uses the measurement data to produce an image that is displayed to a user by way of an operating console 120 and/or stored in a memory unit 121. A central computer unit 122 controls the individual system components.

In MR tomography, images with a high signal to noise ratio (SNR) may be recorded using local coil arrangements (e.g., coils, local coils). The local coil arrangements are antenna systems that are positioned in direct proximity on (anterior), beneath (posterior), next to or in the body 105. During an MR measurement, the excited nuclei induce a voltage in the individual antennas of the local coil. The induced voltage is amplified using a low-noise preamplifier (e.g., LNA, preamp) and is forwarded to the electronic receive system. High-field systems (e.g., 1.5 T-12 T or more) are used to improve the signal to noise ratio, even with high-resolution images. If more individual antennas may be connected to an MR receive system than there are receivers present, a switching matrix (e.g., an RCCS), for example, is incorporated between receive antennas and receivers. This routes the currently active receive channels (e.g., the receive channels in the field of view of the magnet at the time) to the receivers present. This allows more coil elements to be connected than there are receivers present, since for whole body coverage, only the coils present in the FoV or homogeneity volume of the magnet are to be read out.

A local coil arrangement 106 may, for example, be an antenna system that may include one or, in the case of an array coil, a number of antenna elements (e.g., coil elements). The individual antenna elements are embodied, for example, as loop antennas or loops, butterfly coils, flex coils or saddle coils. A local coil arrangement includes, for example, coil elements, a preamplifier, further electronic elements (e.g., baluns), a housing, bearings and may include a cable with plug that is used to connect the local coil arrangement to the MRT system. A receiver 168 on the system side filters and digitizes a signal received by a local coil 106, for example, by radio and transfers the data to a digital signal processing device or facility that may derive an image or spectrum from the data obtained by measurement. The digital signal processing device or facility makes the image or spectrum available to the user for subsequent diagnosis by the user and/or storage.

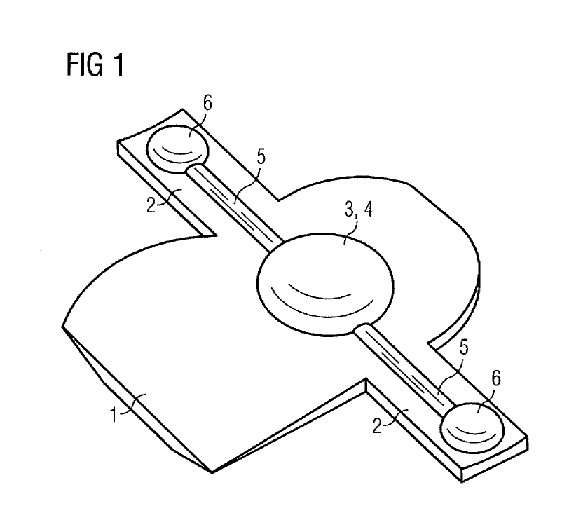

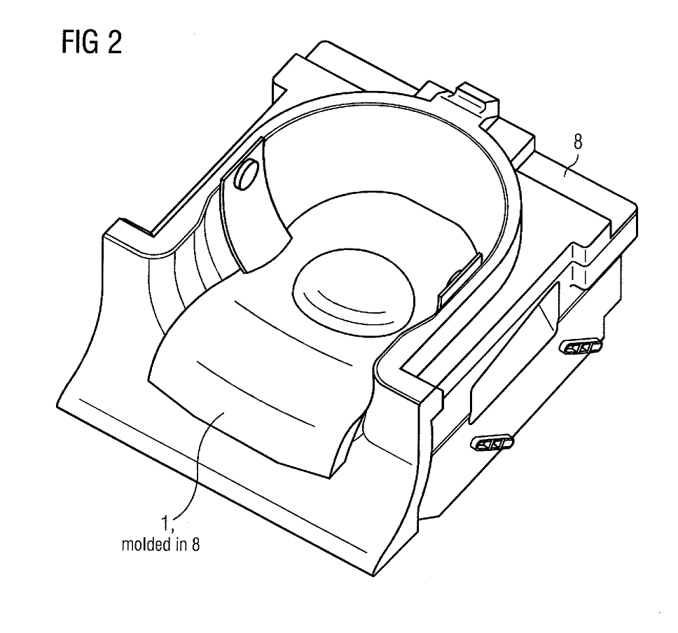

FIG. 1 shows one embodiment of a support cushion 1 to support a body part K (e.g., a head) in a local coil 106. FIGS. 2-4 show one embodiment of a support cushion 1 disposed in one embodiment of an MRT local coil 106 (e.g., bonded or disposed detachably with Velcro).

An MRT local coil 106 may be configured, for example, as a local coil for a head, knee, arm, shoulder or another body part or a number of body parts of a patient 105.

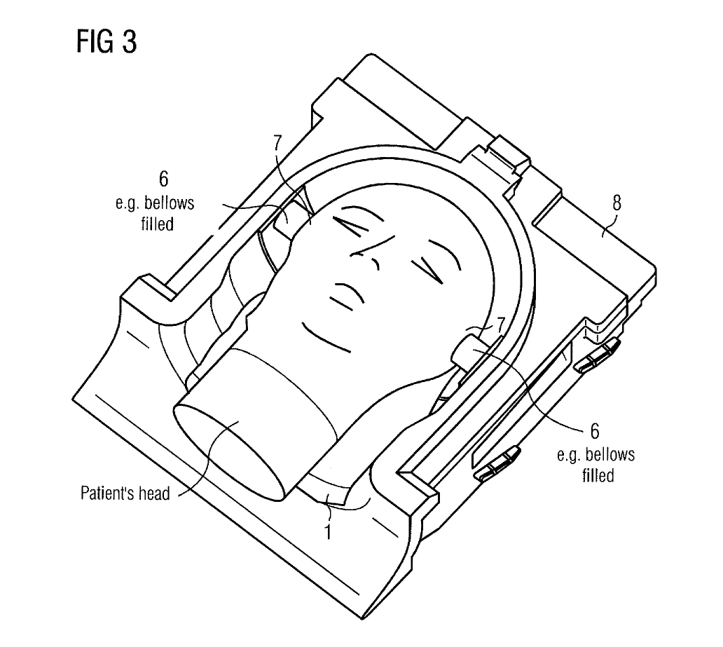

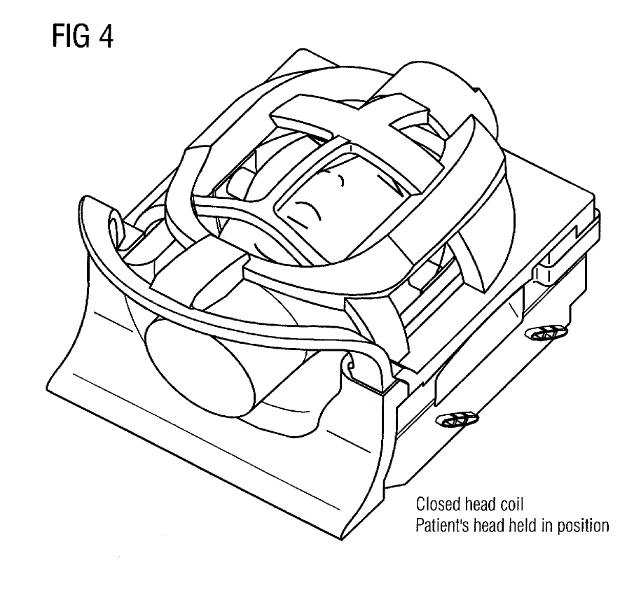

An MRT local coil 106 may have, for example, a local coil lower part 8 illustrated by way of example in FIG. 2 with a cushion 1 therein, with which, after a body part K of a patient 105 has been positioned according, for example, to FIG. 3 on the cushion 1, a local coil upper part according to FIG. 4 is placed on the local coil lower part 8 and may be held in position thereon.

A body part support cushion 1 (e.g., a head support cushion for a head) with an integrated patient positioning system for a body part K of a patient 105 in a local coil with the aid of the (body part) support cushion is described below.

A body part support cushion (e.g., a head support cushion) 1 includes, for example, further elements on the left and right in the form of wings 2. There may be more than two wings for more effective body part positioning. Further elements or wings of the cushion, for example, may be any parts (e.g., hollow parts) of the cushion, (e.g., parts that may be moved to form a body part bearing region of the cushion). In FIG. 2, the wings 2 are bent in and curved to some degree (e.g., towards the coil interior/free space in the coil) compared with the position in FIG. 1 by an inner wall of the local coil and rest against an inner wall of the local coil.

Next to or beneath a bearing region for the body part K (e.g., a head or rear of the head), below a membrane 3, is a volume 4 filled (e.g., completely or partially) with a fluid (e.g., air or another gas or a liquid, such as water; sealed off from the local coil interior; hereafter referred to as the region, the hollow region, or the hollow space).

As the body part K is positioned on the cushion 1, the weight of the body part K causes a fluid (hereafter also referred to as the medium) in the volume 4 of the cushion to be pushed completely or partially out of the volume 4 through channels 5 connected to the volume (e.g., a visible hose as channel 5 but this may also be embedded in the foam of the cushion) into the wing 2. An exchange of fluid takes place, for example, in the form of the movement of fluid from the volume 4 into the elements/wings 2. When the body part is removed from the local coil, an exchange of fluid takes place from one or more elements/wings 2 into the volume 4.

The wings 2 also have a hollow region 6. The region 6 may change volume, for example, using an elastic membrane or, for example, using bellows and is connected to the channels 5.

The elastic region 6 has a greater inherent rigidity or restoring force than the region 3 and/or 4 (e.g., a membrane, boundary or enclosure of the region 3 and/or 4) that may not be extendible (e.g., greater wall thickness in the case of identical material or lower level of elasticity in the case of different materials), so that when the load is removed from the volume 4 (e.g., when the head of a patient leaves the coil), the fluid may return (e.g., automatically) to the volume 4. This is then the starting position for the next patient.

The speed of lateral patient positioning (or the filling of the wings 2) is determined by the cross section of the channels 5. The filled wings 2 may hold the patient in position, for example, at the sides of the forehead 7 or additionally, if there are a number of wings, on one side at other suitable points. Positioning is achieved in that the volume 6 in the wings 2 fills a space on both sides between the coil housing and the body part K (e.g., patient head). The volume 6 is braced against the wall of the head coil lower part 8 (e.g., resting against the wall of the head coil lower part 8). The cushion 1 is molded to a wall in the interior of the coil when the cushion 1 and/or a body part are inserted into the head coil lower part 8.

The wings 2 are pretensioned, so that the wings 2 push out from the local coil interior (e.g., from the local coil center point and/or from a spatial region for a body part to be examined) and do not drop (e.g., automatically) into the interior of the coil (e.g., when there is no body part K therein). The wings may be stiffened with, for example, thin, pliable plastic plates (e.g., approx. 1 mm thick) embedded in the foam of the cushion 1. This type of positioning system may also be used with other volume coils such as neck coils, knee coils, or ankle coils. For any body part (e.g., a head), the positioning system is driven by the weight of the body part to be examined. The lateral wings 2 and the tillable regions 6 may also be embodied to be larger or to be present in larger numbers.

The solution described above causes the head of a patient to be positioned automatically by the insertion of the patient body part K in a local coil (e.g., in a local coil lower part 8). The patient drives the positioning by the weight of the body part of the patient (e.g., the head of the patient), thereby filling the lateral positioning volume 6 in the wings 2. No specific additional handling is required on the part of the operator. If the volume 4 is sufficiently large, the positioning system adjusts to the body part size (e.g., head size). This provides a very good workflow for this point of the examination. Patient comfort is good, as possibly uncomfortable handling around the head may not be provided. When the body part (e.g., the head) is raised, the fluid flows out of the wings 2, the positioning system is released, and the cushion 1 returns to the starting position for the next patient.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A local coil for an imaging system, the local coil comprising:
a support cushion including a volume filled with a fluid.

2. The local coil as claimed in claim 1, wherein at least one element of the support cushion has a region of changeable volume that is connected to the fluid-filled volume of the support cushion.

3. The local coil as claimed in claim 1, wherein the support cushion is a body part support cushion within the local coil for a body part of a patient.

4. The local coil as claimed in claim 1, wherein the local coil is a head coil, and the support cushion is a head support cushion.

5. The local coil as claimed in claim 1, wherein elements of the support cushion each have at least one region with changeable volume and are provided in the form of one or more wings.

6. The local coil as claimed in claim 1, wherein a fluid-filled volume is provided beneath an enclosure or membrane and beneath a bearing region for a body part, within the local coil in a suitable position for imaging in a magnetic resonance tomography (MRT) system at the bottom, or a combination thereof.

7. The local coil as claimed in claim 1, wherein the fluid is or contains a gas, a liquid, or a combination thereof.

8. The local coil as claimed claim 1, wherein the support cushion is configured so that when a body part is positioned on the support cushion, the weight of the body part pushes the fluid completely or partially out of the volume, through channels connected to the volume, into wings of the support cushion.

9. The local coil as claimed in claim 1, wherein the support cushion comprises one or more wings, the one or more wings each having at least one region with changeable volume, the at least one region being connected to one or more channels that are connected to the support cushion, such that a fluid in the support cushion is exchangeable with the one or more channel.

10. The local coil as claimed in claim 1, wherein the support cushion comprises one or more wings, the one or more wings each having a region with changeable volume that is connected to channels that are connected to the fluid-filled volume in a bearing region for a body part.

11. The local coil as claimed in claim 1, wherein the support cushion comprises one or more wings, at least one wing of the one or more wings having a region with changeable volume that has an elastic membrane, a bellows, or a combination thereof, such that a volume of the at least one wing is changeable.

12. The local coil as claimed in claim 11, wherein the region with changeable volume of the at least one wing has a greater inherent rigidity or restoring force than an enclosure or membrane of the volume.

13. The local coil as claimed in claim 1, wherein the support cushion comprises one or more wings, the one or more wings, in a fluid-filled state touching, holding in position, or touching and holding in position a head at sides of the forehead, in particular that a number of wings touch and/or hold in position a body part at one or more points on a number of sides.

14. The local coil as claimed in claim 1, further comprising a positioning system for a body part in the local coil,
wherein one or more wings fill a space between a housing of the local coil and the body part, and
wherein the one or more wings rest against at least one wall of a local coil lower part of the local coil.

15. The local coil as claimed in claim 1, wherein the support cushion contacts, molds to the local coil, or contacts and molds to the local coil when the support cushion is inserted into a coil lower part of the local coil.

16. The local coil as claimed in claim 1, wherein the support cushion comprises wings that are pretensioned in a direction at least partially out from a local coil interior, away from a body part in the local coil, or a combination thereof, in order to prevent the wings from moving automatically in a direction of an interior or an interior of the local coil.

17. The local coil as claimed in claim 1, wherein to establish pretensioning in a direction out from a spatial local coil center point or local coil interior, away from a region of a body part in the local coil, or a combination thereof, wings of the support cushion are stiffened with pliable elements.

18. The local coil as claimed in claim 1, wherein the local coil is a head coil, a neck coil, a knee coil, or an ankle coil.

19. The local coil as claimed in claim 3, wherein the body part is a head.

20. The local coil as claimed in claim 1, wherein the support cushion is disposed in a coil lower part of the local coil.

21. The local coil as claimed in claim 2, wherein the region of changeable volume is for the exchange of fluid.

22. The local coil as claimed in claim 5, wherein at least one wing is provided on the left and right of a position intended for a head.

23. The local coil as claimed in claim 6, wherein the fluid-filled volume is sealed to prevent the egress of fluid into an empty interior of the local.

24. The local coil as claimed in claim 7, wherein the fluid is or contains air or water.

25. The local coil as claimed in claim 12, wherein the region with changeable volume of the at least one wing has a greater wall thickness, a lower elasticity, different or identical material, or a combination thereof to allow fluid to return to the volume when the load is removed from the volume.

26. The local coil as claimed in claim 13, wherein the one or more wings comprise a number of wings that touch, hold in position, or touch and hold in position a body part at one or more points on a number of sides.

27. The local coil as claimed in claim 17, wherein the pliable elements are embedded in foam of the support cushion, and
wherein the pliable elements comprise plastic plates that are more than 0.5 mm and less than 2 mm thick.

28. A support cushion for a local coil, the support cushion comprising:
a volume filled with a fluid,
wherein at least one element of the support cushion has a region of changeable volume that is connected to the fluid-filled volume of the support cushion.
